(19) United States
(12) Patent Application Publication
(10) Pub. No.: US 2015/0018779 A1
(43) Pub. Date: Jan. 15, 2015

(54) SPRING ASSEMBLY FOR A DRUG DELIVERY DEVICE

(71) Applicant: SANOFI-AVENTIS DEUTSCHLAND GMBH, Franfurt am Main (DE)

(72) Inventors: Philippe Nzike, Frankfurt am Main (DE); Michael Schabbach, Frankfurt am Main (DE)

(21) Appl. No.: 14/380,140

(22) PCT Filed: Feb. 20, 2013
(86) PCT No.: PCT/EP2013/053302

§ 371 Date: Aug. 21, 2014

(30) Foreign Application Priority Data
Feb. 22, 2012 (EP) 12156425.6

(51) Int. Cl. A61M 5/20; F16F 3/04

(52) U.S. Cl. CPC A61M 5/2033; F16F 3/04
USPC 604/223; 267/168

(57) ABSTRACT

A spring assembly for a drug delivery device and to such a device, is presented having at least a first coil spring element, and at least a second coil spring element nested arranged with the first coil spring element, and at least a first support member attached to a first end of the first and the second coil spring elements.

### CROSS REFERENCE TO RELATED APPLICATIONS

The present application is a U.S. National Phase Application pursuant to 35 U.S.C. §371 of International Application No. PCT/EP2013/053302 filed Feb. 20, 2013, which claims priority to European Patent Application No. 12156425.6 filed Feb. 22, 2012. The entire disclosure contents of these applications are herewith incorporated by reference into the present application.

### TECHNICAL FIELD

The present invention relates to a spring assembly for a drug delivery device, such as a pen-type injector.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, in particular pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Moreover, a dose setting as well as a dose dispensing procedure must be easy to operate and has to be unambiguous.

Typically, such devices comprise a housing adapted to receive a cartridge at least partially filled with the medicament to be dispensed. The device further comprises a drive mechanism, typically having a displaceable piston rod which is adapted to operably engage with a piston of the cartridge. By means of the drive mechanism and its piston rod, the piston of the cartridge can be displaced in a distal or dispensing direction and may therefore expel a pre-defined amount of the medicament via a piercing assembly which is to be releasably coupled with a distal end section of the housing of the drug delivery device.

A variety of drug delivery devices which may be either manually, automatically or semi-automatically operated comprise a kind of mechanical energy storing mechanism, e.g. in form of at least some spring assembly. By means of a spring assembly, various components of the drug delivery device can be mutually displaced, rotated and/or generally moved e.g. for setting or for dispensing of a dose of the medicament.

For instance, document WO 03/047663 A2 discloses an automatic injector, wherein a needle is automatically injected into an injection site and wherein the needle is retracted after a delivery ends. Prior to and after injection, the needle is withdrawn into a housing of the device to avoid any potential injury or health risk to the user or health care provider. The automatic injector comprises a retracting unit having a spring under compression arranged to push a syringe of the injector away from the injection site. Moreover, a driving unit is provided, which is in communication with a housing and a control unit of the injector to bias the control unit and to pull a piston rod through a barrel.

The driving unit further comprises an extension spring having first and second hooked ends and being attached between the control unit and the housing. The extension spring is attached to the control unit by hooking the first end of the spring into an aperture within the control unit. Accordingly, the driving unit is attached to the housing by hooking its opposite, second end, into a recess fixed to the housing.

Extension springs typically comprise a hook-shaped end in order to attach the spring to a particular object. While the windings of a coiled extension or tension spring provide a rather hollow and tubular outer shape, the hooked ends of such springs typically extend into a middle portion or into a central area of the tubular-shaped spring, thereby limiting the field of application of such springs and their attachment to objects in general.

One possible object of the present invention is to provide an improved spring assembly for use in drug delivery devices. In particular, the spring assembly should provide the general functionality of an extension or tension spring, without being limited to a hooked attachment to components or objects. Moreover, the spring assembly should provide an easy and intuitive attachment and connection to adjoining components and objects and should further provide a large degree of symmetry, in particular, when the spring is tensioned or otherwise elastically deformed.

### SUMMARY

In a first aspect, the present invention provides a spring assembly which is particularly designed for use in a drug delivery device, such like a pen-type injector. The spring assembly provides a mechanical energy storing mechanism and may be universally applicable to displace a selected component of a drug delivery device. For instance, the spring assembly may be adapted to move a needle assembly of a pen-type injector and/or to axially displace a piston rod of a drive mechanism of such drug delivery device, either for a dose setting or for a dose dispensing purpose.

The spring assembly comprises at least a first coil spring element and at least a second coil spring element which is nested arranged with the first coil spring element. Moreover, the spring assembly comprises at least a first support member attached to a first end of the first coil spring and being also attached to a first end of the second coil spring element. Hence, the first support member serves as a common support and fastening structure for both, first and second coil spring elements.

Due to their nested arrangement, first and second coil spring elements are arranged in a substantially overlapping and parallel oriented way. Hence, within the spring assembly, individual windings of first and second coil spring elements are alternately arranged in axial direction, which typically coincides or overlaps with the longitudinal extension of first and second coil spring elements of substantially tubular shape.

Preferably, the at least first support member is provided as a separate piece to be non-releasably interconnected with the first and second coil spring elements. In particular, the at least first support member may be designed and adapted for connecting or engaging the spring assembly with selected components of the drug delivery device.

By providing at least two coil spring elements in a nested, substantially overlapping or mutually penetrating configuration, the spring assembly can be substantially stabilized against lateral forces acting on the spring assembly. Additionally, by having at least two coil spring elements, tension forces provided by the spring assembly are comparatively symmetric and spatially homogenous. In effect, eventual force singularities that may otherwise arise from a hooked connection of a spring assembly to a component or to an object of the drug delivery device, can be effectively avoided or compensated by the at least two-fold interconnection of the at least first support member to the at least first and second coil spring elements.

In a preferred embodiment, the spring assembly further comprises a second support member attached to a second end of the first coil spring element and of the second coil spring element. This way, both opposite ends of first and second coil spring elements are connected and attached to separate support members, preferably in a non-releasable way. The first and second support members may either be substantially identical regarding their geometric shape and size or may differ with respect to each other in order to adapt to a corresponding component of the drug delivery device the first or second support member is to be interconnected with.

In a further preferred embodiment, the first and/or the second support members is or are of substantially annular shape. Preferably, a plane of the annular or circular shaped support member extends substantially perpendicular to the longitudinal axis of first and/or second spring elements. Typically, a surface normal of the plane of the annular shaped first and/or second support members is aligned parallel to the longitudinal central axis of first and/or second coil spring elements and/or substantially coincides or overlaps therewith.

In a further embodiment, the first and/or the second support members of annular or ring-like shape comprise a virtual midpoint substantially coinciding with a cylinder axis or longitudinal axis of the first and/or the second coil spring elements.

Preferably, first and second coil spring elements are substantially identical in size and geometry. Due to their mutually nested arrangement, the longitudinal axes or cylinder axes of first and second coil spring elements substantially overlap. Hence, a virtual midpoint of first and second support members, which may differ in diameter and size, substantially coincides with the cylinder axis of first and second coil spring elements, respectively. As seen in a radial direction, first and second coil spring elements as well as first and second support members are symmetrically arranged around a virtual midpoint or around a virtual cylinder axis of first and/or second coil spring elements.

In a further preferred embodiment, the first and/or second ends of the first and the second coil spring elements are attached at diametrically opposite portions of the first and/or second support members, respectively. Hence, the attachment or fastening points of the first ends of first and second coil spring elements are circumferentially offset by substantially 180°. This way, forces acting on the spring assembly in lateral or radial direction may be more easily compensated by the at least two coil spring elements compared to a conventional spring assembly comprising only one coil spring element.

The at least two-fold attachment of first and second coil spring elements to the at least first and/or second support members therefore provides a rather symmetric and homogenous force distribution in radial direction.

In a further preferred embodiment, the first and/or the second coil spring elements are bonded with the first and/or with the second support members. The coil spring element and the support members may be adhesively joined or may be mutually bonded, preferably by means of a thermally induced deformation or formation of the first and/or second support member. For instance, the first and/or the second support members may comprise a plastic component adaptable to become non-releasably interconnected with both, first and second coil spring elements. In principle, the first and/or second support members can be provided as injection molded components being directly molded to first and/or second ends of first and/or second coil spring elements.

In another preferred embodiment, the first and/or the second support members comprise a thermally treatable plastic material to thermally engage with the first end and/or with the second end of the first and/or second coil spring element. For instance, the support member may comprise at least two through openings or recesses to receive a corresponding end portion of the first and the second coil spring element. During manufacture of the spring assembly, the respective end portions of first and second coil spring elements can be inserted into said through openings or recesses.

By thermally treating, in particular by heating the plastic material of first and/or second support members, a firmly mutual bonding of the support member with first and second coil spring elements can be attained. A mutual engagement and bonding of first and second coil spring elements with either one of first or second support members can be provided by e.g. laser-or ultrasonic welding, wherein a well-defined amount of thermal energy can be deposited on or in the respective support member.

In a further embodiment, an end winding or initial winding of the at least first and/or second coil spring element directly abuts with the first and/or with the second support member, respectively. This way, a rather large and circumferential axial abutment configuration between first and second coil spring element and first and/or second support members can be provided. This way, the spring assembly becomes less prone to radially directed tilting motions when subject to a lateral or radially directed force effect.

Additionally, by means of a comparatively large direct abutment between an initial or end winding of first and/or second coil spring elements and the adjacently disposed first or second support member, a radially or transversally directed force effect acting on e.g. first or second support members can be directly transferred to the first and second spring elements, which in response may generate respective counter-directed forces.

Preferably, the end winding or initial winding of the first and/or the second coil spring element axially abuts along at least 15°, 30°, 45°, 60° or even along at least 90° with the first and/or second support member in tangential or circumferential direction.

In a further preferred embodiment, the at least first and second coil spring elements are of substantially equal axial length and comprise substantially equal diameters. Moreover, the at least two spring elements may also comprise substantially equal spring constants. In order to modify the spring constant of the entire spring assembly, it is also conceivable, that first and second coil spring elements comprise different spring constants to provide a required or predefined total spring constant of the spring assembly.

In still another aspect, the outer diameter of the at least one support member may be larger than the outer diameter of the first and second coil spring elements. This way, the spring assembly can be easily arranged, e.g. in a tubular housing or receptacle of the drug delivery device, wherein the radially widened support members can be interconnected with selected device components, e.g. with a radial recess while the at least first and second coil spring elements remain substantially contactless and frictionless with regard to the surrounding housing or receptacle of the drug delivery device.

Moreover, by providing a first and/or second annular support member, the spring assembly, in particular it's at least first and second coil spring elements may also receive a tubular shaped component of the drug delivery device extending there through. Such components, when comprising a diameter smaller than the inner diameter of first and second spring element, may not only extend through the at least first and second coil spring elements but may also extend through the first and/or second support member. Therefore, the present spring assembly also provides a space-saving arrangement of mechanical components of a drug delivery device and allows to reduce overall dimensions and geometry of such drug delivery devices and their drive mechanisms.

Naturally, the spring assembly is not limited to only two coil spring elements. In a preferred embodiment, the spring assembly comprises at least three or even more coil spring elements nested arranged with respect to each other and being attached to the at least first and/or second support members. Typically the first, the second and the at least third coil spring elements are arranged in an overlapping or nested configuration such that individual windings of first, second and third coil spring elements alternate in axial direction.

With three coil spring elements for instance, respective fastening or attachment points of first, second and third coil spring elements to e.g. any one of first or second support members are circumferentially offset by 120°. Preferably, the attachment or fastening point of the various coil spring elements to either the first or second support member are equidistantly arranged in tangential or circumferential direction, on order to improve the symmetry of a radial force distribution across the spring assembly.

Even though not explicitly mentioned, in the present context, any reference made to first and second coil spring elements is to be understood to equally apply to a multitude of e.g. three, four or even more coil spring elements to be arranged in a nested way between first and second support members.

In a further preferred aspect the at least first and second spring elements comprise at least one extension spring element. However, the present invention is not generally limited to tension spring assemblies but may also refer to a compression spring assembly. Additionally, it is also conceivable, that the at least first spring element comprises an extension spring while the at least second spring element comprises a compression spring. This way, the resulting spring constant of the entire spring assembly may be individually adapted to predefined requirements.

In another independent aspect, the invention also relates to a drug delivery device and in particular to a pen-type injector. The device comprises a housing to accommodate a cartridge of vial- or carpule-type and being filled with a medicament. Furthermore, the housing is also adapted to accommodate a drive mechanism having inter alia a piston rod to operably engage with a piston of said cartridge. The cartridge is typically provided as a vial or carpule and may be removably disposed in the housing of the drug delivery device in order to frequently replace a used cartridge by a new and filled one.

Alternatively, the drug delivery device may be of disposable type and may be intended to be entirely discarded once the medicament provided in the cartridge has been used up. The drug delivery device further comprises at least one spring assembly as described above for spring loading at least one selected component of the drive mechanism of the drug delivery device relative to another component thereof. The spring element may be further operable for spring loading an axially displaceable needle assembly of an automatic injector with regard to the housing of such a device.

The at least one spring assembly is generally adapted or applicable to bias any two components of the drug delivery device that are to be spring loaded. Since the at least first support member of the spring assembly can be individually adapted to appropriately engage with a selected component of the drug delivery device and/or of its drive mechanism, the present spring assembly can be universally adapted to various different geometric requirements of the drug delivery device and any of its spring loaded components.

In a further preferred embodiment, the drug delivery device comprises at least one receptacle or recess to receive the at least one support member of the spring assembly. The receptacle or recess of the drug delivery device therefore substantially matches and mates with the correspondingly shaped and radially protruding support member of the spring assembly in order to provide a well-defined mutual arrangement of the spring assembly and the particular component of the drug delivery device.

In still another embodiment, the drug delivery device further comprises a cartridge being at least partially filled with the medicament which is to be administered and/or to be dispensed by means of the drug delivery device.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39); or
des Pro36 [Asp28] Exendin-4(1-39),
des Pro36 [IsoAsp28] Exendin-4(1-39),
des Pro36 [Met(O)14, Asp28] Exendin-4(1-39),
des Pro36 [Met(O)14, IsoAsp28] Exendin-4(1-39),
des Pro36 [Trp(O2)25, Asp28] Exendin-4(1-39),
des Pro36 [Trp(O2)25, IsoAsp28] Exendin-4(1-39),
des Pro36 [Met(O)14 Trp(O2)25, Asp28] Exendin-4(1-39),
des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
des Pro36 Exendin-4(1-39)-Lys6-NH2 (AVE0010),
H-(Lys)6-des Pro36 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro36 [Trp(02)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro36 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(0)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro36 [Met(O)14, Trp(O2)25, Asp28] Exendin-4(1-39)-Lys6-NH2,
H-des Asp28 Pro36, Pro37, Pro38 [Met(O)14, Trp(02)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
   or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, preferred embodiments of the invention will be described in detail by making reference to the drawings, in which:

FIG. 1 schematically illustrates a side view of a spring assembly comprising a first and a second coil spring element arranged in a nested configuration,

FIG. 2 shows a cross section through the spring assembly according to FIG. 1 along A-A,

FIG. 3 shows a cross section through the spring assembly along B-B,

FIG. 4 is illustrative of the spring assembly according to FIG. 1 in a released, non-extended configuration and

FIG. 5 exemplary illustrates a drug delivery device in form of a pen-type injector in an exploded view.

### DETAILED DESCRIPTION

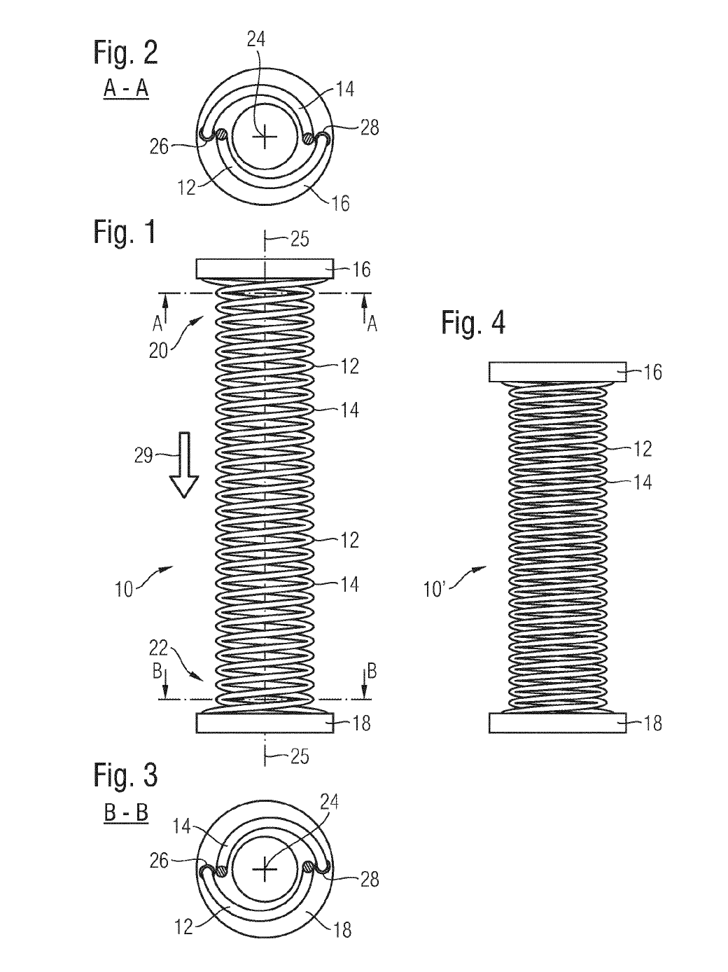

The extended spring assembly 10 as illustrated in FIG. 1 comprises a first coil spring element 12 and a second coil spring element 14, which are arranged in a nested or substantially overlapping configuration. First and second spring elements 12, 14 comprise substantially equal diameters and substantially equal axial length along a longitudinal central axis 25 as indicated in FIG. 1. According to the mutually nested configuration of the first coil spring element 12 and the second coil spring element 14, the individual windings of first and second coil spring elements 12, 14 are alternately arranged in axial direction, hence along the longitudinal axis 25.

With an upper or first end 20, the first coil spring element 12 as well as the second coil spring element 14 is non-releasably attached to a first support member 16 as illustrated in FIG. 2. With their opposite second end 22, the two coil spring elements 12, 14 are correspondingly attached to a second annular shaped support member 18, as illustrated in FIG. 3.

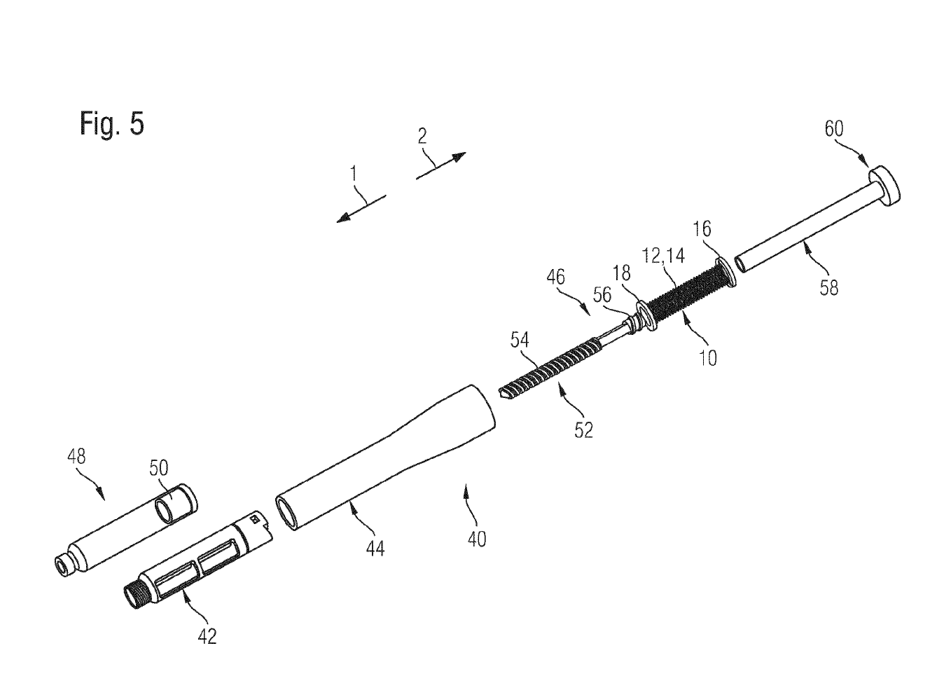

As further shown in FIGS. 1 to 3, the outer circumference of the annular shaped support members 16, 18 is slightly larger than the outer circumference of the first and second coil spring elements 12, 14. As depicted in FIGS. 2 and 3, the inner diameter of first and second support members 16, 18 is substantially equal to the inner diameter of first and second coil spring elements 12, 14, which are arranged in a flushed configuration with the annular shaped first and second support members 16, 18. This way, a functional component of a drug delivery device 40 as shown in FIG. 5 may extend through the spring assembly 10 as well as through the support members 16, 18.

The first and second coil spring elements 12, 14 as well as first and second support members 16, 18 are substantially symmetrically arranged with respect to the central longitudinal axis 25 of first and second coil spring elements 12, 14. Hence, a virtual midpoint 24 of the first and the second support members 16, 18 substantially coincides and overlaps with the longitudinal axis 25.

As becomes further apparent from FIGS. 2 and 3, the attachment of the first coil spring element 12 and the attachment of the second coil spring element with first and second support members 16, 18 is diametrically opposite. Preferably, the first and second support members 16, 18 are provided with a recess or through hole 26, 28 which is adapted to receive an end portion of the first and second coil spring elements 12, 14, respectively.

By thermally treating, especially by heating the first and second support members appropriately, a firm bonding of first and second coil spring elements 12, 14 with first and second support members 16, 18 can be provided. Since the at least first and second coil spring elements 12, 14 comprise a metal, e.g. an annealed metal wire, it is only the first and/or second support member as a plastic component that can be locally melted by way of a thermal treatment for non-releasably engaging first and second coil spring elements 12, 14 and first and/or second support members 16, 18.

Generally, the spring assembly 10 as shown in an expanded configuration in FIG. 1 can be adapted to and applied in various different configurations and embodiments with a drug delivery device as only exemplary illustrated in FIG. 5. Preferably, the spring assembly 10 is provided as an extension spring, wherein the oppositely located first and second support members 16, 18 may positively engage with components of a drug delivery device 40 that have to be spring loaded with respect to each other.

As shown in FIG. 4, the spring assembly 10′ comprises an initial and substantially relaxed configuration, in which individual windings of first and second coil spring elements 12, 14 are in direct abutment or axial contact with each other. When extending the spring assembly 10 into an extended configuration as shown in FIG. 1, the first and second extension spring elements 12, 14 provide a restoring force 29 as indicated by the arrow in FIG. 1, that aims to bring together first and second support members 16, 18 in axial or longitudinal direction.

The drug delivery device 40 as shown in FIG. 5 is a pen-type injector and comprises two housing components, namely a distally arranged cartridge holder 42 which is adapted to accommodate a cartridge 48 being at least partially filled with a medicament and being sealed in proximal direction 2 by means of a piston 50 slidably displaced therein. The drug delivery device 40 further comprises a body or main housing component 44 which is adapted to accommodate a drive mechanism 46 having at least a piston rod 52 which is adapted to operably engage with the piston 50 of the cartridge 48.

The drive mechanism 46 further comprises a drive sleeve 58 threadedly engaged with a proximal outer thread 56 of the piston rod 52. Additionally, the drive sleeve 58 comprises a dose dial member 60, by way of which a user can set and/or dispense a dose of the medicament which is provided in the cartridge 48. In distal direction 1, the piston rod 52 comprises a second outer thread 54 which cooperates with an inner thread of the main housing 44, which is not explicitly illustrated here.

By way of a rotational movement of the piston rod 52 relative to the housing 44, the piston rod 52 moves in distal direction 1, thereby correspondingly advancing the piston 50 of the cartridge 48. The distally located threaded socket of the cartridge holder 42 is further adapted to releasably receive a needle assembly having a double-tipped needle that is adapted to penetrate a distally located seal of the cartridge 48. Having a needle correctly mounted on the cartridge holder 42, distally directed displacement of the piston 50 relative to the barrel of the cartridge 48 leads to a well-defined dispensing or injecting of the medicament through the needle assembly.

As further shown in FIG. 5, the drive mechanism 46 also comprises a spring assembly 10 as illustrated in FIGS. 1 to 4. Here, a proximally located support member 16 engages with the drive sleeve 58 in a rotation invariant way. For this purpose, the drive sleeve 58 and/or the dose dial member 60 may comprise a radial recess to receive the support member 16. Accordingly, the opposite support member 18 of the spring assembly 10 may radially engage with an inside located recess of the main housing 44, thereby fixing the support member 18 in axial direction relative to the housing 44 while the support member 18 may remain free to rotate relative to the housing 44.

By means of the spring assembly 10 acting as an extension spring, the entire drive mechanism 46 or selected components thereof can be spring loaded either mutually or with respect to the housing 44.

1-15. (canceled)

16. A spring assembly for a drug delivery device, the spring assembly comprising:
at least a first coil spring element, and
at least a second coil spring element nested arranged with the first coil spring element, and
at least a first support member attached to a first end of the first and the second coil spring elements, characterized in that the first support member is of annular shape and comprises a thermally treatable plastic material to firmly engage with the first end of the first and second coil spring elements and wherein the at least first and second coil spring elements are of equal axial length and comprise equal diameters.

17. The spring assembly according to claim 16, further comprising a second support member attached to a second end of the first and the second coil spring elements.

18. The spring assembly according to claim 17, wherein the second support member is of annular shape.

19. The spring assembly according to claim 18, wherein the first and/or the second support members comprise a virtual midpoint coinciding with a cylinder axis of the first and second coil spring elements.

20. The spring assembly according to claim 16, wherein the first and/or second ends of the first and the second coil spring elements are attached at diametrically opposite portions of the first and/or second support members, respectively.

21. The spring assembly according to claim 16, wherein the first and/or the second coil spring elements are bonded with the first and/or second support members.

22. The spring assembly according to claim 16, wherein the second support member comprise a thermally treatable plastic material to firmly engage with the second end of the first and second coil spring elements.

23. The spring assembly according to claim 16, wherein an end winding of the at least first and/or second coil spring elements directly abuts with the first and/or second support member, respectively.

24. The spring assembly according to claim 16, wherein the at least first and the second coil spring elements comprise an equal amount of windings.

25. The spring assembly according to claim 16, wherein the outer diameter of the at least one support member is larger than the outer diameter of the first and second coil spring elements.

26. The spring assembly according to claim 16, further comprising at least three or more coil spring elements nested arranged with respect to each other and being attached to the at least first and/or second support members.

27. The spring assembly according to claim 16, wherein the at least first and second spring elements comprise an extension spring element.

28. A drug delivery device for administering a dose of a medicament, the device comprising:
a housing to accommodate a cartridge filled with a medicament and to accommodate a drive mechanism having a piston rod to operably engage with a piston of said cartridge, and
at least one spring assembly according to claim 16, for spring loading at least one selected component of the drive mechanism relative to another component of the drive mechanism or relative to the housing.

29. The drug delivery device according to claim 28, further comprising at least one receptacle or recess to receive the at least one support member of the spring assembly.

30. The drug delivery device according to claim 28, further comprising a cartridge at least partially filled with a medicament.
